(19) United States
(12) Patent Application Publication
(10) Pub. No.: US 2022/0081575 A1
(43) Pub. Date: Mar. 17, 2022

(54) SUBSTRATE, SELECTIVE FILM DEPOSITION METHOD, DEPOSITION FILM OF ORGANIC MATTER, AND ORGANIC MATTER

(71) Applicant: CENTRAL GLASS COMPANY, LIMITED, Yamaguchi (JP)

(72) Inventors: Masutaka SHINMEN, Yamaguchi (JP); Takuya OKADA, Yamaguchi (JP); Junki YAMAMOTO, Yamaguchi (JP); Ryo NADANO, Yamaguchi (JP); Tatsuo MIYAZAKI, Tokyo (JP)

(73) Assignee: CENTRAL GLASS COMPANY, LIMITED, Yamaguchi (JP)

(21) Appl. No.: 17/421,507

(22) PCT Filed: Jan. 7, 2020
(86) PCT No.: PCT/JP2020/000171

§ 371 Date: Jul. 8, 2021

(30) Foreign Application Priority Data
Jan. 10, 2019 (JP) 2019-002313
Apr. 24, 2019 (JP) 2019-083110

(51) Int. Cl. C09D 7/20; C07C 211/03; B05D 1/00

(52) U.S. Cl. CPC C09D 7/20; B05D 1/60; C07C 211/03

(57) ABSTRACT

The selective film deposition method according to an embodiment of the present disclosure includes depositing a film of an organic matter represented by the following formula (1) on a substrate having a structure where a first surface region containing at least one of a metal or a metal oxide and a second surface region containing a nonmetallic inorganic material are both exposed, selectively in the first surface region than in the second surface region,

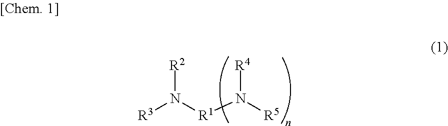

wherein N represents a nitrogen atom; and R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms.

### TECHNICAL FIELD

The present disclosure relates to a substrate, a selective film deposition method for selectively depositing a film in a surface region containing at least one of a metal or a metal oxide on a substrate, a deposition film of an organic matter, and an organic matter.

### BACKGROUND ART

Recent semiconductor chips have more minute structures to raise problems such as the high number of steps and high cost in the production thereof by conventional lithography in which patterning is carried out by selectively removing part of the structure. The above problems are considered to be overcome by formation of a film selectively in a desired part on a substrate by chemical vapor deposition (CVD) or atomic layer deposition (ALD), which is an optimal process for formation of a minute structure.

In a case where a film is selectively deposited by CVD or ALD on a substrate including multiple surface regions different in the type of metal used for electrodes or wiring and the type of material of inorganic dielectrics used for insulating films, a film for inhibiting deposition needs to be selectively deposited. In conventional methods, however, the selectivity is not high enough.

A known technique for selective formation of a film is deposition of a material that inhibits deposition of a film in a region where a film is not desired to be formed. Patent Literature 1, for example, discloses a method for forming a film pattern of an inorganic material such as TiN, AlN, or SiN on a substrate by atomic layer deposition (ALD), the method including: forming a pattern of an atomic layer deposition-inhibiting layer on a substrate by screen printing or the like using an atomic layer deposition-inhibiting material prepared from a fluororesin that has a fluorine content of 30 atom % or higher, contains at least one tertiary carbon atom or quaternary carbon atom, and has no ester, hydroxyl, carboxyl, or imide groups; and forming an inorganic material layer by atomic layer deposition in a region where the atomic layer deposition-inhibiting layer is not present.

Patent Literature 2 discloses a method for selectively depositing a layer atop a substrate having an exposed metal surface and an exposed silicon-containing surface, the method including: (a) growing a first self-assembled monolayer atop the exposed metal surface; (b) growing an organosilane-based second self-assembled monolayer atop the exposed silicon-containing surface; (c) heating the substrate to remove the first self-assembled monolayer from atop the exposed metal surface; (d) selectively depositing a layer atop the exposed metal surface, wherein the layer is a low-k dielectric layer or a metal layer; and (e) heating the substrate to remove the second self-assembled monolayer from atop the exposed silicon-containing surface.

According to the above methods, on a substrate having first and second surfaces containing different materials, a film can be deposited selectively on the first surface than on the second surface, utilizing the difference in surface state of the two surfaces. Moreover, according to the above methods, the number of steps included in the process for forming a minute structure can be reduced.

Patent Literature 3, for example, discloses a process for depositing an organic film on a substrate including a first surface that is a metallic surface and a second surface that is a dielectric surface, selectively on the first surface than on the second surface, the process including a deposition cycle including: contacting the substrate with a first gaseous precursor; and contacting the substrate with a second gaseous precursor. In Example 1 of Patent Literature 3, a polyimide film was formed on a substrate that was a 200-mm silicon wafer including tungsten (W) features alternating with silicon oxide surfaces by 250 to 1000 deposition cycles using 1,6-diaminohexane (DAH) and pyromellitic dianhydride (PMDA). In this case, the polyimide film on a metallic tungsten surface was thicker than the polyimide film on a SiO₂ surface.

Patent Literature 4 discloses a method utilizing the selective deposition method of an organic film of Patent Literature 3 for selectively forming a passivation layer on the metallic first surface and then forming a layer X only on the dielectric second surface, and also discloses a method utilizing the foregoing method for forming a metallization structure of an integrated circuit.

### CITATION LIST

### Patent Literature

Patent Literature 1: WO2016/147941A1
Patent Literature 2: JP 2018-512504 T
Patent Literature 3: JP 2017-216448 A
Patent Literature 4: JP 2018-137435 A

### SUMMARY OF INVENTION

### Technical Problem

Patent Literature 1, however, only discloses a method for forming a predetermined pattern on a substrate formed of a single material using an atomic layer deposition-inhibiting material, not disclosing a method for forming an atomic layer deposition-inhibiting layer selectively in a desired surface region on a substrate including multiple surface regions containing different materials.

The organosilane self-assembled monolayer in Patent Literature 2 is deposited selectively on a silicon-containing surface and cannot be deposited selectively on a metal or metal oxide.

The methods for selectively forming organic films according to Patent Literature 3 and Patent Literature 4 include repetition of the deposition cycle more than once in which the raw material and the temperature are changed, which requires a great deal of time and effort.

In consideration of the above problems, the present disclosure aims to provide a selective film deposition method for selectively depositing a film of an organic matter in a surface region containing at least one of a metal or a metal oxide than in a surface region containing a nonmetallic inorganic material on a substrate by simple operation, and a deposition film of an organic matter deposited by the method, and the organic matter.

### Solution to Problem

The present inventors made intensive studies to find out that use of an organic matter represented by the formula (1) mentioned later enables deposition of a film of an organic matter selectively in a surface region containing at least one of a metal or a metal oxide than in a surface region containing a nonmetallic inorganic material on a substrate. Thus, the present disclosure was completed.

A selective film deposition method according to an embodiment of the present disclosure includes a step of depositing a film of an organic matter represented by the following formula (1) on a substrate having a structure where a first surface region containing at least one of a metal or a metal oxide and a second surface region containing a nonmetallic inorganic material are both exposed, selectively in the first surface region than in the second surface region,

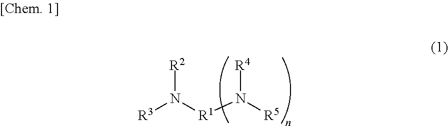

wherein N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

According to the selective film deposition method, use of an organic matter represented by the formula (1) enables deposition of a film of an organic matter selectively in a first surface region containing at least one of a metal or a metal oxide exposed on a substrate than in a second surface region containing a nonmetallic inorganic material exposed on the substrate by simple operation.

A substrate according to an embodiment of the present disclosure has a structure where a first surface region containing at least one of a metal or a metal oxide and a second surface region containing a nonmetallic inorganic material are both exposed. The substrate includes a film of an organic matter represented by the formula (1) mentioned above in the first surface region. The substrate includes no film of the organic matter in the second surface region or includes a film of the organic matter having a thickness t₂ smaller than a thickness t₁ of the film of the organic matter in the first surface region.

According to the substrate, a film of an organic matter is deposited selectively in a first surface region containing at least one of a metal or a metal oxide exposed on the substrate than in a second surface region containing a nonmetallic inorganic material exposed on the substrate.

A deposition film of an organic matter according to an embodiment of the present disclosure is a deposition film of an organic matter formed by the above method. The organic matter selectively deposited on a substrate is represented by the formula (1) mentioned above.

An organic matter according to an embodiment of the present disclosure is an organic matter used in the method for depositing a film selectively in a surface region containing at least one of a metal or a metal oxide of a substrate. The organic matter is represented by the formula (1) mentioned above.

Use of the organic matter enables deposition of a film of the organic matter selectively in the first surface region containing at least one of a metal or a metal oxide exposed on the substrate than in the second surface region containing a nonmetallic inorganic material exposed on the substrate by simple operation.

A solution according to an embodiment of the present disclosure contains an organic matter represented by the formula (1) mentioned above and a solvent.

### Advantageous Effects of Invention

According to the selective film deposition method according to the embodiment of the present disclosure, provided is a method in which use of an organic matter represented by the formula (1) mentioned above enables deposition of a film of the organic matter represented by the formula (1) selectively in a first surface region containing at least one of a metal or a metal oxide exposed on a substrate than in a second surface region containing a nonmetallic inorganic material exposed on the substrate by simple operation.

According to a substrate according to the embodiment of the present disclosure, provided is a substrate in which a film of an organic matter represented by the formula (1) is deposited selectively in a first surface region containing at least one of a metal or a metal oxide exposed on the substrate than in a second surface region containing a nonmetallic inorganic material exposed on the substrate.

### DESCRIPTION OF EMBODIMENTS

The present disclosure is described in detail below. The following description of structural elements provides exemplary embodiments of the present disclosure. The present disclosure is not limited to these specific embodiments. Various modifications can be made within the scope of the gist.

A selective film deposition method according to an embodiment of the present disclosure includes depositing a film of an organic matter represented by the formula (1) mentioned above on a substrate having a structure where a first surface region containing at least one of a metal or a metal oxide and a second surface region containing a nonmetallic inorganic material are both exposed, selectively in the first surface region than in the second surface region.

According to the method, use of an organic matter represented by the formula (1) enables deposition of a film of an organic matter selectively in a first surface region containing at least one of a metal or a metal oxide exposed on a substrate than in a second surface region containing a nonmetallic inorganic material exposed on the substrate. At this time, preferably, a film of the organic matter is selectively deposited only in the first surface region and not deposited in the second surface region. Alternatively, a film of the organic matter having a thickness t₂ smaller than the thickness t₁ of the film of the organic matter in the first surface region is deposited in the second surface region preferably in a manner that the ratio t₁/t₂ obtained by dividing t₁ by t₂ is 5 or higher. The ratio t₁/t₂ is preferably 10 or higher, more preferably 100 or higher.

Deposition of a film (hereafter, also referred to as deposition film) of an organic matter can be determined by dropping pure water on the surface of the substrate and measuring the angle (contact angle) between the droplet and the substrate surface with a contact angle meter.

Specifically, when an organic matter represented by the formula (1) which is poorly compatible with water covers the substrate surface, the contact angle with water is large.

In the selective film deposition method according to the embodiment of the present disclosure, the contact angle with water in the first surface region is larger than that in the second surface region by preferably 10° or more, more preferably 20° or more, still more preferably 30° or more.

Thus, it can be determined that a film of an organic matter is selectively deposited in the first surface region having a large contact angle with water than in the second surface region having a small contact angle with water.

Whether or not a deposition film of an organic matter is formed on a substrate can be also determined by analysis of the elemental composition of the substrate surface by X-ray photoelectron spectroscopy (XPS). In a case where the organic matter contains a characteristic atom such as nitrogen, the peak of that element can be observed.

The metal may be at least one selected from the group consisting of Cu, Co, Ru, Ni, Pt, Al, Ta, Ti, and Hf. The metal oxide may be an oxide of at least one metal selected from the group consisting of Cu, Co, Ru, Ni, Pt, Al, Ta, Ti, and Hf. In particular, the metal is preferably Cu, Co, or Ru and the metal oxide is preferably an oxide of Cu, Co, or Ru. The metal and metal oxide each may be a mixture of these metals or metal oxides. The metal may also be an alloy and the metal oxide may be a natural surface oxide film of the metal or an alloy containing the metal.

Examples of the nonmetallic inorganic material contained in the second surface region include silicon materials such as silicon, silicon oxides, silicon nitrides, and silicon oxynitrides and germanium materials such as germanium, germanium oxides, germanium nitrides, and germanium oxynitrides. Preferred are silicon materials among these nonmetallic inorganic materials. The term “silicon” herein refers to both polycrystalline silicon and monocrystalline silicon. The silicon oxides are represented by the formula SiOₓ (x is 1 or larger and 2 or smaller) and a typical example thereof is SiO₂. The silicon nitrides are represented by SiNₓ (x is 0.3 or larger and 9 or smaller) and a typical example thereof is Si₃N₄. The silicon oxynitrides are represented by Si₄OₓN_{y} (x is 3 or larger and 6 or smaller and y is 2 or larger and 4 or smaller) and an example thereof is Si₄O₅N₃.

The first surface region in which a metal is exposed is obtained, for example, by forming a metallic film by chemical vapor deposition (CVD) or physical vapor deposition (PVD). For example, a metallic film is formed on a film of the nonmetallic inorganic material and the metallic film is patterned in a predetermined pattern by photolithography. Alternatively, holes or grooves are formed in a film of the nonmetallic inorganic material and the holes or grooves are filled with a metal. Thus, a substrate having a structure where a first surface region containing a metal and a second surface region containing a nonmetallic inorganic material are both exposed can be obtained.

The first surface region in which a metal is exposed can be also obtained by removing a surface oxide film of a metallic film with a solution containing HF or the like to expose a metal surface. The oxide film may also be mechanically removed.

The first surface region in which a metal oxide is exposed can be obtained by forming a film of a metal oxide by CVD or PVD. Alternatively, it may be obtained by exposing a metal film preliminarily obtained by a similar method to the air to form a natural oxide film. For example, a film of a metal oxide is formed on a film of the nonmetallic inorganic material and the film of a metal oxide is patterned in a predetermined pattern by photolithography. Alternatively, holes or grooves are formed in a film of the nonmetallic inorganic material and the holes or grooves are filled with a metal, followed by formation of a natural oxide film on the metal. Thus, a substrate having a structure where a first surface region containing a metal oxide and a second surface region containing a nonmetallic inorganic material are both exposed can be obtained.

The first surface region containing at least one of a metal or a metal oxide may additionally contain a different compound on which an organic matter represented by the formula (1) can be deposited other than the metal and the metal oxide, or consist of at least one of a metal or a metal oxide. Preferably, the first surface region consists of at least one of a metal or a metal oxide and the at least one of a metal or a metal oxide alone is exposed on the surface.

The second surface region containing a nonmetallic inorganic material may contain a compound other than the nonmetallic inorganic material, or consist of a nonmetallic inorganic material. Preferably, the second surface region consists of a nonmetallic inorganic material and the nonmetallic inorganic material alone is exposed on the surface.

Examples of the substrate used in the embodiment of the present disclosure include a substrate of a semiconductor device including a metal or metal oxide film in the structure and a substrate on which a metal or metal oxide film is formed during the patterning process of a semiconductor device. In particular, preferred is a substrate prepared by forming metal wiring in a predetermined pattern on an insulating film of a semiconductor element. Specifically, metal wiring having a natural surface oxide film and metal-exposed metal wiring correspond to the first surface region. An insulating film formed of a nonmetallic inorganic material corresponds to the second surface region. The substrate used in the embodiment of the present disclosure is not limited to these.

A film of an organic matter represented by the formula (1) is deposited selectively in the first surface region than in the second surface region specifically by the following two methods: a method of exposing the substrate to a solution containing an organic matter and a solvent (wet method); and a method of exposing the substrate to an atmosphere containing an organic matter in a gaseous state (dry method). These methods are described in the following.

### [Wet Method]

In the wet method according to the embodiment of the present disclosure, a substrate is exposed to a solution containing the organic matter and a solvent. In an exemplary film deposition process, a substrate including a first surface region and a second surface region is immersed in a solution containing an organic matter and a solvent to bring the solution into contact with the substrate surface, thereby depositing a film of the organic matter selectively in the first surface region of the substrate. The exposure of a substrate to a solution refers to contact of a substrate surface with a solution. Accordingly, examples of the method for exposing a substrate to a solution include, in addition to the immersion method, spin coating in which a solution is dropped onto a substrate and the substrate is spun fast and spray coating in which a solution is sprayed to a substrate. The method may be any method that can bring a substrate into contact with a solution.

The concentration of the organic matter in the solution based on the total of the organic matter and the solvent is preferably 0.01% by mass or higher and 20% by mass or lower, preferably 0.1% by mass or higher and 10% by mass or lower, more preferably 0.5% by mass or higher and 8% by mass or lower, particularly preferably 1% by mass or higher and 5% by mass or lower. In the case where the solution contains multiple types of organic matters, the concentration range indicated above refers to the total concentration of the organic matters.

The organic matter used in the wet method is an organic matter represented by the following formula (1).

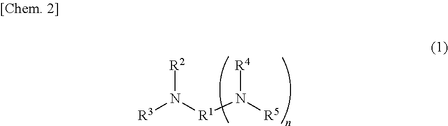

(In the formula (1), N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.)

Examples of the hetero atom optionally contained in the hydrocarbon group for R¹ to R⁵ include a nitrogen atom, an oxygen atom, a sulfur atom, and a phosphorus atom. Examples of the halogen atom include a fluorine atom, a chlorine atom, a bromine atom, and an iodine atom. In the case where the carbon number is 3 or more, the hydrocarbon group may be a branched hydrocarbon group such as an isopropyl group or a tert-butyl group, an aromatic hydrocarbon group such as a phenyl group, or an alicyclic hydrocarbon group such as a cyclohexyl group free from a non-aromatic conjugated double bond. In the case where R³ and R⁵ both contain 1 or more carbon atoms, they may directly combine with each other to form a macrocyclic structure such as a porphyrin ring for the formula (1). R², R³, R⁴, and R⁵ may represent the same hydrocarbon group or different hydrocarbon groups.

Examples of R², R³, R⁴, and R⁵ include a hydrogen group and a hydrocarbon group. R² and R³ each preferably represent a hydrogen group (hydrogen atom). R², R³, R⁴, and R⁵ may all represent a hydrogen atom. In that case, the organic matter represented by the formula (1) is diamine.

The organic matter represented by the formula (1) may be represented by the formula (1) wherein n represents 0, R² and R³ each represent a hydrogen group, and R¹ represents a phenyl group or a cyclohexyl group. Preferably, R¹ represents a C1-C30 hydrocarbon group optionally containing a hetero atom or a halogen atom. More preferably, R¹ represents a C1-C20 alkyl group.

Among these, the organic matter represented by the formula (1) is preferably an amino group (—NH₂)-containing organic matter represented by the formula (1) wherein R² and R³ each represent a hydrogen atom. Examples of the organic matter include methylamine, ethylamine, n-propylamine, n-butylamine, n-pentylamine, n-hexylamine, n-heptylamine, n-octylamine, n-nonylamine, n-decylamine, n-undecylamine, n-dodecylamine, n-tridecylamine, n-tetradecylamine, n-pentadecylamine, n-hexadecylamine, margarylamine (i.e., n-heptadecylamine), stearylamine (i.e., n-octadecylamine), n-nonadecylamine, phenylamine, (2-phenylethyl)amine, (3-phenylpropyl)amine, (4-phenylbutyl)amine, methylenediamine, (4-aminophenyl)amine, (4-aminobenzyl)amine, cyclohexylamine, aniline, benzhydrylamine, (4-bromophenyl)amine, (2-chloroethyl)amine, (3-chloropropyl)amine, (4-chlorobutyl)amine, (5-chloropentyl)amine, (6-chlorohexyl)amine, (2-bromoethyl)amine, (3-bromopropyl)amine, (4-bromobutyl)amine, (5-bromopentyl)amine, (6-bromohexyl)amine, ethylenediamine, 1,3-propylenediamine, 1,4-butanediamine, 1,5-pentanediamine, 1,6-hexylenediamine, 1,4-phenylenediamine, o-xylylenediamine, m-xylylenediamine, p-xylylenediamine, (aminomethyl)amine, (1-aminoethyl)amine, 2-(perfluorobutyl)ethylamine, 2-(perfluorohexyl)ethylamine, and 2-(perfluoroheptyl)ethylamine.

A primary amine represented by the formula (1) wherein n represents 0 and containing one amino group is preferred because it is inexpensive and contains one amino group in the compound to be less likely to form a film containing an amino group that is not combined with the first surface region of the substrate.

A linear alkylamine represented by the formula (1) wherein n represents 0, and R¹ represents a C1-C30 linear hydrocarbon group optionally containing a hetero atom or a halogen atom, and containing one amino group can form a favorable deposition film. In particular, R¹ preferably represents a C6-C24 alkyl group, more preferably C8-C20 alkyl group. Examples of such an organic matter include n-octylamine, n-nonylamine, n-decylamine, n-undecylamine, n-dodecylamine, n-tridecylamine, n-tetradecylamine, n-pentadecylamine, n-hexadecylamine, margarylamine, and stearylamine.

The solvent used in the solution of the present disclosure may be any conventionally known solvent that can dissolve the above organic matter and is not likely to cause damage to the surface to be treated. From the standpoint of solubility of the organic matter and less damage to the surface to be treated, preferred is an organic solvent other than water (nonaqueous solvent). From the standpoint of solubility of the organic matter, preferred is a nonaqueous solvent other than a hydrocarbon solvent.

The nonaqueous solvent other than a hydrocarbon solvent is suitably, for example, an ester, an ether, a ketone, a sulfoxide solvent, a sulfone solvent, a lactone solvent, a carbonate solvent, an alcoholic solvent, a derivative of a polyhydric alcohol, a nitrogen element-containing solvent, a silicone solvent, or a mixture of these. Moreover, the nonaqueous solvent used is preferably an ester, an ether, a ketone, an alcoholic solvent, or a derivative of a polyhydric alcohol.

Examples of the ester include ethyl acetate, n-propyl acetate, i-propyl acetate, n-butyl acetate, i-butyl acetate, n-pentyl acetate, i-pentyl acetate, n-hexyl acetate, n-heptyl acetate, n-octyl acetate, n-pentyl formate, n-butyl propionate, ethyl butyrate, n-propyl butyrate, i-propyl butyrate, n-butyl butyrate, methyl n-octanoate, methyl decanoate, methyl pyruvate, ethyl pyruvate, n-propyl pyruvate, methyl acetoacetate, ethyl acetoacetate, ethyl 2-oxobutanoate, dimethyl adipate, 3-methoxymethyl propionate, 3-methoxyethyl propionate, 3-ethoxymethyl propionate, 3-ethoxyethyl propionate, and ethyl ethoxyacetate.

Examples of the ether include di-n-propyl ether, ethyl-n-butyl ether, di-n-butyl ether, ethyl-n-amyl ether, di-n-amyl ether, ethyl-n-hexyl ether, di-n-hexyl ether, di-n-octyl ether, ethers containing a branched hydrocarbon group corresponding to the carbon number of the foregoing ethers, such as diisopropyl ether and diisoamyl ether, dimethyl ether, diethyl ether, methyl ethyl ether, methyl cyclopentyl ether, diphenyl ether, tetrahydrofuran, dioxane, methyl perfluoropropyl ether, methyl perfluorobutyl ether, ethyl perfluorobutyl ether, methyl perfluorohexyl ether, and ethyl perfluorohexyl ether.

Examples of the ketone include acetone, acetyl acetone, methyl ethyl ketone, methyl propyl ketone, methyl butyl ketone, 2-heptanone, 3-heptanone, cyclohexanone, and isophorone.

Examples of the sulfoxide solvent include dimethyl sulfoxide. Examples of the sulfone solvent include dimethyl sulfone, diethyl sulfone, bis(2-hydroxyethyl)sulfone, and tetramethylene sulfone.

Examples of the lactone solvent include β-propiolactone, γ-butyrolactone, γ-valerolactone, γ-hexanolactone, γ-heptanolactone, γ-octanolactone, γ-nonanolactone, γ-decanolactone, γ-undecanolactone, γ-dodecanolactone, δ-valerolactone, δ-hexanolactone, δ-octanolactone, δ-nonanolactone, δ-decanolactone, δ-undecanolactone, δ-dodecanolactone, and ε-hexanolactone.

Examples of the carbonate solvent include dimethyl carbonate, ethyl methyl carbonate, diethyl carbonate, and propylene carbonate. Examples of the alcoholic solvent include methanol, ethanol, propanol, butanol, pentanol, hexanol, heptanol, octanol, ethylene glycol, diethylene glycol, 1,3-propanediol, 1,2-propanediol, dipropylene glycol, 1,2-butanediol, 1,3-butanediol, 1,4-butanediol, triethylene glycol, tripropylene glycol, tetraethylene glycol, tetrapropylene glycol, and glycerin.

Examples of the derivative of a polyhydric alcohol include: OH group-containing polyhydric alcohol derivatives such as ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutyl ether, triethylene glycol monomethyl ether, triethylene glycol monoethyl ether, triethylene glycol monopropyl ether, triethylene glycol monobutyl ether, tetraethylene glycol monomethyl ether, tetraethylene glycol monoethyl ether, tetraethylene glycol monopropyl ether, tetraethylene glycol monobutyl ether, propylene glycol monomethyl ether, propylene glycol monoethyl ether, propylene glycol monopropyl ether, propylene glycol monobutyl ether, dipropylene glycol monomethyl ether, dipropylene glycol monoethyl ether, dipropylene glycol monopropyl ether, dipropylene glycol monobutyl ether, tripropylene glycol monomethyl ether, tripropylene glycol monoethyl ether, tripropylene glycol monopropyl ether, tripropylene glycol monobutyl ether, tetrapropylene glycol monomethyl ether, and butylene glycol monomethyl ether; and OH group-free polyhydric alcohol derivatives such as ethylene glycol dimethyl ether, ethylene glycol diethyl ether, ethylene glycol dibutyl ether, ethylene glycol monomethyl ether acetate, ethylene glycol monoethyl ether acetate, ethylene glycol monobutyl ether acetate, ethylene glycol diacetate, diethylene glycol dimethyl ether, diethylene glycol ethyl methyl ether, diethylene glycol diethyl ether, diethylene glycol butyl methyl ether, diethylene glycol dibutyl ether, diethylene glycol monomethyl ether acetate, diethylene glycol monoethyl ether acetate, diethylene glycol monobutyl ether acetate, diethylene glycol diacetate, triethylene glycol dimethyl ether, triethylene glycol diethyl ether, triethylene glycol dibutyl ether, triethylene glycol butyl methyl ether, triethylene glycol monomethyl ether acetate, triethylene glycol monoethyl ether acetate, triethylene glycol monobutyl ether acetate, triethylene glycol diacetate, tetraethylene glycol dimethyl ether, tetraethylene glycol diethyl ether, tetraethylene glycol dibutyl ether, tetraethylene glycol monomethyl ether acetate, tetraethylene glycol monoethyl ether acetate, tetraethylene glycol monobutyl ether acetate, tetraethylene glycol diacetate, propylene glycol dimethyl ether, propylene glycol diethyl ether, propylene glycol dibutyl ether, propylene glycol monomethyl ether acetate, propylene glycol monoethyl ether acetate, propylene glycol monobutyl ether acetate, propylene glycol diacetate, dipropylene glycol dimethyl ether, dipropylene glycol methyl propyl ether, dipropylene glycol diethyl ether, dipropylene glycol dibutyl ether, dipropylene glycol monomethyl ether acetate, dipropylene glycol monoethyl ether acetate, dipropylene glycol monobutyl ether acetate, dipropylene glycol diacetate, tripropylene glycol dimethyl ether, tripropylene glycol diethyl ether, tripropylene glycol dibutyl ether, tripropylene glycol monomethyl ether acetate, tripropylene glycol monoethyl ether acetate, tripropylene glycol monobutyl ether acetate, tripropylene glycol diacetate, tetrapropylene glycol dimethyl ether, tetrapropylene glycol monomethyl ether acetate, tetrapropylene glycol diacetate, butylene glycol dimethyl ether, butylene glycol monomethyl ether acetate, butylene glycol diacetate, glycerin triacetate, 3-methoxybutyl acetate, 3-methyl-3-methoxybutyl acetate, and 3-methyl-3-methoxybutyl propionate.

Examples of the nitrogen element-containing solvent include N,N-dimethylformamide, N,N-dimethylacetamide, N,N-diethylacetamide, N-methyl-2-pyrrolidone, N-ethyl-2-pyrrolidone, N-propyl-2-pyrrolidone, 1,3-dimethyl-2-imidazolidinone, 1,3-diethyl-2-imidazolidinone, 1,3-diisopropyl-2-imidazolidinone, triethylamine, and pyridine.

Examples of the silicone solvent include hexamethyl disiloxane, octamethyl trisiloxane, decamethyl tetrasiloxane, and dodecamethyl pentasiloxane.

From the standpoint of solubility of the organic matter, the organic solvent is preferably a polar organic solvent. In particular, an alcoholic solvent is preferred. For example, ethanol or isopropyl alcohol (IPA) is favorably used.

The solvent may be blended with water. In this case, the water concentration based on 100% by mass of the solution of the present disclosure is preferably 40% by mass or lower, particularly preferably 20% by mass or lower, still more preferably 10% by mass or lower.

For the purpose of promoting formation of a deposition film of the organic matter, the solution of the present disclosure may contain a catalyst such as an acidic compound (e.g., hexafluoroisopropanol, trifluoroacetic acid, anhydrous trifluoroacetic acid, trifluoromethanesulfonic acid, anhydrous trifluoromethanesulfonic acid) and a basic compound (e.g., pyridine, N,N-dimethyl-4-aminopyridine, ammonia, imidazole). The amount of the catalyst for 100% by mass of the total amount of the solution is 0.01 to 50% by mass.

The solution temperature during the film deposition process by the wet method is preferably 0° C. to 80° C. The immersion time of the substrate in the solution is preferably 10 seconds or longer and 48 hours or shorter, preferably 1 minute or longer and 24 hours or shorter. The immersion time may be 1 second or longer and 1000 seconds or shorter. Upon immersion of the substrate in the solution, the solution is preferably stirred with a stirring blade or the like.

After exposure of the substrate to the solution containing the organic matter, a washing step in which the substrate is washed with a solvent is preferably carried out. Examples of the solvent usable in the washing step include the above-mentioned organic solvents. The substrate is preferably washed by immersion in the solvent at 0° C. to 80° C. for 1 to 1000 seconds. In the case where the substrate is immersed in the solution containing the organic matter, the substrate is taken out from the solution and then washed with a solvent.

After the washing step, an inert gas such as nitrogen or argon gas is preferably blown to the substrate so that the substrate is dried. The temperature of the inert gas blown is preferably 0° C. to 80° C.

### [Dry Method]

In the dry method according to the embodiment of the present disclosure, the substrate is exposed to an atmosphere containing an organic matter in a gaseous state. Specifically, in the film deposition step, the substrate is placed in a chamber, a gas containing an organic matter is introduced into the chamber to bring the gas containing an organic matter into contact with the substrate surface, and a film of the organic matter is deposited selectively in the first surface region of the substrate.

The organic matter used in the film deposition step by the dry method is an organic matter represented by the formula (1) as in the wet method.

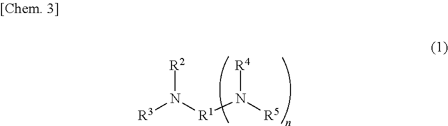

(In the formula (1), N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.)

In the formula (1) representing the organic matter used in the dry method, examples of the hetero atom optionally contained in the hydrocarbon group for R¹ to R⁵ include a nitrogen atom, an oxygen atom, a sulfur atom, and a phosphorus atom. In the case where R³ and R⁵ both contain 1 or more carbon atoms, they may directly combine with each other to form a macrocyclic structure such as a porphyrin ring for the formula (1). R², R³, R⁴, and R⁵ may represent the same hydrocarbon group or different hydrocarbon groups.

The organic matter represented by the formula (1) may be an organic matter represented by the formula (1) wherein n represents 0, R² and R³ each represent a hydrogen atom, and R¹ represents a C3-C10 hydrocarbon group, a phenyl group, or a cyclohexyl group. Alternatively, the organic matter represented by the formula (1) may be a diamine represented by the formula (1) wherein n represents 1 and R² to R⁴ each represent a hydrogen group, or a dialkyl amine represented by the formula (1) wherein n represents 0, R² represents a hydrogen atom, and R¹ and R³ each represent a hydrocarbon group containing 1 or more carbon atoms.

For deposition of a film having a sufficient thickness, the organic matter represented by the formula (1) is preferably an amino group (—NH₂)-containing organic matter represented by the formula (1) wherein R² and R³ each represent a hydrogen atom. Examples of the organic matter include n-butylamine, n-pentylamine, n-hexylamine, n-heptylamine, n-octylamine, cyclohexylamine, aniline, ethylenediamine, and 2-aminoethanol.

In particular, a primary amine represented by the formula (1) wherein n represents 0 and containing one amino group, such as n-butylamine, n-pentylamine, n-hexylamine, n-heptylamine, n-octylamine, cyclohexylamine, or aniline, is preferred because it is inexpensive and contains one amino group in the compound to be less likely to form a film containing an amino group that is not combined with the substrate.

The atmosphere gas in the chamber containing the organic matter in a gaseous state has a temperature of preferably 0° C. or higher and 200° C. or lower, more preferably 5° C. or higher and 100° C. or lower, particularly preferably 10° C. or higher and 80° C. or lower.

The atmosphere gas in the chamber containing the organic matter in a gaseous state has a pressure range of 0.1 Torr (13 Pa) or higher and 500 Torr (67 kPa) or lower, more preferably 1 Torr (0.13 kPa) or higher and 100 Torr (13 kPa) or lower.

In order to bring the organic matter in a gaseous state into contact with the substrate, the temperature and pressure inside the chamber need to be set to allow the organic matter remain in a gaseous state.

The atmosphere gas in the chamber contains the organic matter in a gaseous state in an amount of 1 vol % or more and 100 vol % or less, more preferably 10 vol % or more and 100 vol % or less, still more preferably 50 vol % or more and 100 vol % or less. The organic matter in a gaseous state may be obtained by decompressing and/or heating the organic matter in a liquid state. Alternatively, the organic matter in a liquid state may be bubbled with an inert gas to obtain the organic matter in a gaseous state diluted with an inert gas. The inert gas used may be nitrogen gas, argon gas, krypton gas, or neon gas.

The organic matter in a gaseous state may be obtained by decompressing and/or heating the organic matter in a liquid state. Alternatively, the organic matter in a liquid state may be bubbled with an inert gas to obtain the organic matter in a gaseous state diluted with an inert gas. The inert gas used may be nitrogen gas, argon gas, krypton gas, or neon gas.

Excessive organic matter can be removed by decompressing the chamber to 1 to 100 Pa after the film deposition step by the dry method. The dry method does not have to include a drying step.

Employment of the wet method or the dry method according to the embodiment of the present disclosure enables deposition of a film of an organic matter selectively in a surface region in which at least one of a metal or a metal oxide is exposed than in a surface region in which a nonmetallic inorganic material is exposed on a substrate by simple operation.

A deposition film of an organic matter represented by the formula (1) selectively deposited on a substrate by the wet method or the dry method corresponds to an embodiment of the deposition film of an organic matter of the present disclosure.

[Substrate after Selective Deposition of Deposition Film of Organic Matter]

The substrate according to an embodiment of the present disclosure is a substrate having a structure where a first surface region containing at least one of a metal or a metal oxide and a second surface region containing a nonmetallic inorganic material are both exposed, the substrate including a film of an organic matter represented by the following formula (1) in the first surface region, the substrate including no film of the organic matter in the second surface region or including a film of the organic matter having a thickness t₂ smaller than a thickness t₁ of the film of the organic matter in the first surface region.

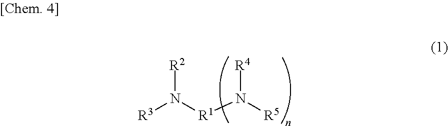

(In the formula (1), N represents a nitrogen atom; R¹ represents a C1-C30 hydrocarbon group optionally containing a hetero atom or a halogen atom, R², R³, R⁴, and R⁵ each represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.)

As described above, the substrate includes a film of an organic matter represented by the above formula (1) in the first surface region, and includes no film of the organic matter in the second surface region or includes a film of the organic matter having a thickness t₂ smaller than a thickness t₁ of the film of the organic matter in the first surface region.

In the case where the thickness t₂ of the film of the organic matter in the second surface region is smaller than the thickness t₁ of the film of the organic matter in the first surface region on the substrate, the value of t₁/t₂ obtained by dividing t₁ by t₂ is preferably 5 or larger. The ratio of t₁/t₂ is preferably 10 or larger, more preferably 100 or larger. The thickness t₁ is preferably 0.3 nm or larger, preferably 0.6 nm or larger, preferably 1 nm or larger, more preferably 2 nm or larger, still more preferably 3 nm or larger. The thickness t₂ is preferably smaller than 1 nm, preferably smaller than 0.3 nm and may be 0 nm. The thicknesses t₁ and t₂ can be measured with an atomic force microscope (AFM). The thickness t₂ of 0 nm means satisfaction of the above condition. Specifically, the film of the organic matter is selectively deposited only in the first surface region.

The first surface region containing at least one of a metal or a metal oxide, the second surface region containing a nonmetallic inorganic material, and the organic matter represented by the formula (1) in the substrate according to the embodiment of the present disclosure are not specifically described here as they are already described in the selective film deposition method according to the embodiment of the present disclosure in which a film is selectively deposited in the first surface region of the substrate.

The film of an organic matter is considered to be formed by interaction of a group having a nitrogen atom, an oxygen atom, or a sulfur atom in the molecule of the organic matter with the metal or metal oxide in the first surface region.

The present disclosure also encompasses an organic matter represented by the formula (1) used in the selective film deposition method of the present disclosure. The present disclosure further encompasses a solution containing the organic matter and the solvent.

### EXAMPLES

Selective deposition of a film of an organic matter in a surface region in which a metal or a metal oxide is exposed was confirmed by the following experiments.

### Experimental Example 1-1

In isopropyl alcohol (hereafter, referred to as IPA) was dissolved n-dodecylamine at a concentration of 1%. Thus, a solution containing n-dodecylamine as an organic matter and a solvent was prepared.

In the solution was immersed a substrate including a Cu natural oxide film for 60 seconds so that a film of an organic matter was deposited. The solution temperature was 20° C. to 25° C. Then, the substrate was immersed in an IPA liquid at 20° C. to 25° C. for 60 seconds twice for removal of excessive organic matter. To the substrate was blown nitrogen gas at 20° C. to 25° C. for 60 seconds so that the substrate was dried.

The thickness of the film of an organic matter formed on the substrate measured with an atomic force microscope (AFM) was 3 nm. In the elemental composition analyzed by X-ray photoelectron spectroscopy (XPS), a strong nitrogen peak was observed.

### Experimental Examples 1-2 to 1-16

Experimental examples were carried out and evaluated as in Experimental Example 1-1, except that the type of the metal oxide on the substrate surface, the type of the organic matter, the type of the solvent, and the solution concentration (concentration of the organic matter), and the like were changed as shown in Table 1. Table 1 shows the results.

TABLE 1

| | | | | Solution | | Surface |
|---|---|---|---|---|---|---|
| | | | | concentration | Thickness | elemental |
| | Surface | Organic matter | Solvent | (%) | (nm) | composition |
| Experimental Example 1-1 | | n-Dodecylamine | IPA | 1 | 3 | N |
| Experimental Example 1-2 | | Stearylamine | IPA | 1 | 4 | N |
| Experimental Example 1-3 | | Cyclohexylamine | IPA | 1 | 2 | N |
| Experimental Example 1-4 | Cu oxide film | Aniline | IPA | 1 | 2 | N |
| Experimental Example 1-5 | | n-Dodecylamine | Ethanol | 1 | 3 | N |
| Experimental Example 1-6 | | Stearylamine | Ethanol | 1 | 4 | N |
| Experimental Example 1-7 | | Cyclohexylamine | Ethanol | 1 | 1 | N |
| Experimental Example 1-8 | | Aniline | Ethanol | 1 | 1 | N |
| Experimental Example 1-9 | | n-Dodecylamine | IPA | 1 | 3 | N |
| Experimental Example 1-10 | | Stearylamine | IPA | 1 | 4 | N |
| Experimental Example 1-11 | | Cyclohexylamine | IPA | 1 | 1 | N |
| Experimental Example 1-12 | Co oxide film | Aniline | IPA | 1 | 2 | N |
| Experimental Example 1-13 | | n-Dodecylamine | Ethanol | 1 | 3 | N |
| Experimental Example 1-14 | | Stearylamine | Ethanol | 1 | 4 | N |
| Experimental Example 1-15 | | Cyclohexylamine | Ethanol | 1 | 1 | N |
| Experimental Example 1-16 | | Aniline | Ethanol | 1 | 1 | N |

### Experimental Example 2-1

In IPA was dissolved n-dodecylamine at a concentration of 5%. Thus, a solution containing n-dodecylamine as an organic matter and a solvent was prepared.

In the solution was immersed a substrate including a Si surface as a nonmetallic inorganic material for 60 seconds so that a film of an organic matter was deposited. The solution temperature was 20° C. to 25° C. Then, the substrate was immersed in an IPA liquid at 20° C. to 25° C. for 60 seconds twice for removal of excessive organic matter. To the substrate was blown nitrogen gas at 20° C. to 25° C. for 60 seconds so that the substrate was dried.

The thickness of the film of an organic matter formed on the substrate measured with an AFM was 0 nm. In the elemental composition analyzed by XPS, no nitrogen peak was observed.

### Experimental Examples 2-2 to 2-8

Experimental examples 2-2 to 2-8 were carried out and evaluated as in Experimental Example 2-1, except that the type of the nonmetallic inorganic material on the substrate surface, the type of the organic matter, the type of the solvent, and the solution concentration (concentration of the organic matter) were changed as shown in Table 2. Table 2 shows the results.

TABLE 2

| | | | | Solution | | Surface |
|---|---|---|---|---|---|---|
| | | | | concentration | Thickness | elemental |
| | Surface | Organic matter | Solvent | (%) | (nm) | composition |
| Experimental Example 2-1 | Si | n-Dodecylamine | IPA | 5 | 0 | — |
| Experimental Example 2-2 | Si | Stearylamine | IPA | 5 | 0 | — |
| Experimental Example 2-3 | SiO₂ | n-Dodecylamine | IPA | 5 | 0 | — |
| Experimental Example 2-4 | SiO₂ | Stearylamine | IPA | 5 | 0 | — |
| Experimental Example 2-5 | SiN | n-Dodecylamine | IPA | 5 | 0 | — |
| Experimental Example 2-6 | SiN | Stearylamine | IPA | 5 | 0 | — |
| Experimental Example 2-7 | SiON | n-Dodecylamine | IPA | 5 | 0 | — |
| Experimental Example 2-8 | SiON | Stearylamine | IPA | 5 | 0 | — |

In the above experimental examples, the Cu natural oxide film (Cu oxide film)-containing substrate was obtained by forming a copper film on a silicon substrate by vapor deposition to a thickness of about 100 nm and exposing the resulting substrate to the air.

The Co natural oxide film (Co oxide film)-containing substrate was obtained by forming a cobalt film on a silicon substrate by vapor deposition to a thickness of about 100 nm and exposing the resulting substrate to the air.

The Si surface-containing substrate was obtained by removing a natural oxide film on a silicon substrate.

The SiO₂ surface-containing substrate was obtained by forming a silicon dioxide film on a silicon substrate by chemical vapor deposition to a thickness of about 30 nm.

The SiN surface-containing substrate was obtained by forming a silicon nitride film represented by the formula Si₃N₄ on a silicon substrate by chemical vapor deposition to a thickness of about 30 nm.

The SiON surface-containing substrate was obtained by oxidizing a SiN surface formed on a silicon substrate and forming a silicon oxinitride film represented by the formula Si₄OₓN_{y} (x is 3 or larger and 6 or smaller, y is 2 or larger and 4 or smaller) by chemical vapor deposition to a thickness of about 10 nm.

### Experimental Example 3-1

In a vacuum processing chamber was placed a substrate including a CuO surface, and the chamber pressure was set to 15 Torr (2.0 kPa absolute pressure). Next, a cylinder containing ethylenediamine connected to the chamber was set to be warmed at 20° C. and the valve was opened to supply ethylenediamine in a gaseous state to the chamber. Thus, the CuO-containing substrate was brought into contact with ethylenediamine in a gaseous state so that a film of an organic matter was deposited on the substrate. The temperature of the chamber was the same as the cylinder temperature. The temperature of ethylenediamine in a gaseous state was maintained at the same temperature as the cylinder warming temperature until being brought into contact with the substrate. After deposition of the film of an organic matter, the chamber was decompressed to 1 Torr (0.13 kPa) for removal of excessive organic matter.

The thickness of the film of an organic matter formed on the substrate measured with an AFM was 8 nm. In the elemental composition analyzed by XPS, a strong nitrogen peak was observed.

### Experimental Examples 3-2 to 3-16

Experimental examples 3-2 to 3-16 were carried out and evaluated as in Experimental Example 3-1, except that the type of the metal oxide on the substrate, the type of the organic matter, the cylinder warming temperature (organic matter heating temperature), and the chamber pressure (absolute pressure) were changed as shown in Table 3. Table 3 shows the results.

TABLE 3

| | | | Organic | Absolute | Thick- |
|---|---|---|---|---|---|
| | Target | Organic | matter heating | pressure | ness |
| | surface | matter | temperature (° C.) | (Torr) | (nm) |
| Experimental Example 3-1 | CuO | Ethylenediamine | 20 | 15 | 8 |
| Experimental Example 3-2 | CuO | n-Butylamine | 20 | 50 | 5 |
| Experimental Example 3-3 | CuO | n-Hexylamine | 30 | 10 | 5 |
| Experimental Example 3-4 | CuO | n-Octylamine | 50 | 5 | 4 |
| Experimental Example 3-5 | CuO | Cyclohexylamine | 30 | 10 | 3 |
| Experimental Example 3-6 | CuO | Aniline | 60 | 5 | 3 |
| Experimental Example 3-7 | CuO | Di-n-butylamine | 50 | 10 | 1 |
| Experimental Example 3-8 | CuO | 2-Aminoethanol | 50 | 5 | 4 |
| Experimental Example 3-9 | CoO | Ethylenediamine | 20 | 15 | 10 |
| Experimental Example 3-10 | CoO | n-Butylamine | 20 | 50 | 6 |
| Experimental Example 3-11 | CoO | n-Hexylamine | 30 | 10 | 5 |
| Experimental Example 3-12 | CoO | n-Octylamine | 50 | 5 | 4 |
| Experimental Example 3-13 | CoO | Cyclohexylamine | 30 | 10 | 3 |
| Experimental Example 3-14 | CoO | Aniline | 60 | 5 | 3 |
| Experimental Example 3-15 | CoO | Di-n-butylamine | 50 | 10 | 1 |
| Experimental Example 3-16 | CoO | 2-Aminoethanol | 50 | 5 | 4 |

### Experimental Example 4-1

In a vacuum processing chamber was placed a substrate including a Si surface as a nonmetallic inorganic material, and the chamber pressure was set to 15 Torr. Next, a cylinder containing ethylenediamine connected to the chamber was set to be warmed at 20° C. and the valve was opened. Thus, the Si surface-containing substrate was brought into contact with ethylenediamine in a gaseous state. After deposition of a film of an organic matter, the chamber was decompressed to 0.1 Torr for removal of excessive organic matter.

The thickness of the film of an organic matter formed on the substrate measured with an AFM was 0 nm. In the elemental composition analyzed by XPS, no nitrogen peak was observed.

### Experimental Examples 4-2 to 4-10

Experimental examples 4-2 to 4-10 were carried out and evaluated as in Experimental Example 4-1, except that the type of the nonmetallic inorganic material on the substrate, the cylinder warming temperature (organic matter heating temperature), and the chamber pressure (absolute pressure) were changed as shown in Table 4. Table 4 shows the results.

TABLE 4

| | | | Organic | Absolute | |
|---|---|---|---|---|---|
| | Target | | matter heating | pressure | Thickness |
| | surface | Organic matter | temperature (° C.) | (Torr) | (nm) |
| Experimental Example 4-1 | Si | Ethylenediamine | 20 | 15 | 0 |
| Experimental Example 4-2 | Si | n-Hexylamine | 30 | 10 | 0 |
| Experimental Example 4-3 | Si | Cyclohexylamine | 30 | 10 | 0 |
| Experimental Example 4-4 | Si | Aniline | 60 | 5 | 0 |
| Experimental Example 4-5 | Si | 2-Aminoethanol | 50 | 5 | 0 |
| Experimental Example 4-6 | SiO₂ | Ethylenediamine | 20 | 15 | 0 |
| Experimental Example 4-7 | SiO₂ | n-Hexylamine | 30 | 10 | 0 |
| Experimental Example 4-8 | SiO₂ | Cyclohexylamine | 30 | 10 | 0 |
| Experimental Example 4-9 | SiO₂ | Aniline | 60 | 5 | 0 |
| Experimental Example 4-10 | SiO₂ | 2-Aminoethanol | 50 | 5 | 2 |

In Experimental Examples 3-1 to 3-16 and 4-1 to 4-10, the CuO surface-containing substrate was obtained by forming a copper oxide film on a silicone substrate by vapor deposition to a thickness of about 100 nm.

The CoO surface-containing substrate was obtained by forming a cobalt oxide film on a silicon substrate by vapor deposition to a thickness of about 100 nm.

The Si surface-containing substrate was obtained by removing a natural oxide film of a silicon substrate.

The SiO₂ surface-containing substrate was obtained by forming a silicon dioxide film on a silicon substrate by chemical vapor deposition to a thickness of about 30 nm.

As clearly seen from the results in Tables 1 to 4, in the experimental examples, the organic matter was deposited on the metal oxide surface such as CuO (Cu oxide film) and CoO (Co oxide film) surfaces but not deposited on the nonmetallic inorganic material surface such as Si, SiO₂, SiN, and SiON surfaces. Accordingly, the experimental examples show that, in the case of using a substrate including a surface region in which a metal oxide is exposed and a surface region in which a nonmetallic inorganic material is exposed, use of the organic matters shown in Tables 1 to 4 enables selective deposition of a film only in the surface region in which a metal oxide is exposed.

In particular, in the wet method according to Tables 1 and 2, use of n-dodecylamine or stearylamine represented by the formula (1) in which R¹ is a linear alkyl group enabled deposition of a film having a thickness of 3 nm or larger. In the case of using cyclohexylamine or aniline represented by the formula (1) in which R¹ has a cyclic structure, the film formed was as thin as 1 to 2 nm.

In the dry method according to Tables 3 and 4, particularly in Experimental Examples 3-1 to 3-6 and Experimental Examples 3-9 to 3-14, the film formed had a thickness of 3 nm or larger owing to use of ethylenediamine that is a primary amine containing two amino groups or n-butylamine, n-hexylamine, n-octylamine, cyclohexylamine, or aniline that is a primary amine containing one amino group. In contrast, in Experimental Examples 3-7 and 3-15 in which di-n-butylamine that is a secondary amine was used, the film formed was very thin.

In Experimental Examples 4-5 and 4-10 in which 2-aminoethanol containing an amino group and a hydroxyl group (OH group) was used, the film was deposited not on Si but on SiO₂. In other words, the selectivity to the metal oxide surface than to the SiO₂ surface was not good. As seen from the results of Experimental Examples 3-1 to 3-7, 3-9 to 3-15, 4-1 to 4-4, and 4-6 to 4-9, use of an organic matter represented by the formula (1) containing amino group(s) alone enabled deposition of a film of an organic matter selectively in the first surface region containing a metal oxide than in the second surface region in both the case where the second surface region contained Si and the case where the second surface region contained SiO₂.

### Experimental Example 5-1

### (Preparation of Solution)

n-Octadecylamine as an organic matter was blended with isopropyl alcohol (IPA) as a solvent and dissolved therein at a concentration of the organic matter of 1% by mass. Thus, a solution containing n-dodecylamine as an organic matter and a solvent was prepared.

### (Preparation of Substrate)

The surface of a silicon substrate including a 100-nm-thick cobalt film was oxidized by UV/03 irradiation (lamp: EUV200WS, distance to lamp: 10 mm, ozone is generated from oxygen in the air by UV irradiation) for 30 minutes. Thus, a substrate containing cobalt oxide (CoOx) on the surface was obtained.

(Surface Treatment with Solution Containing Organic Matter)

The substrate was immersed in the solution at 22° C. for 24 hours for surface treatment of the substrate. Thus, the organic matter was deposited on the substrate surface. Then, the substrate was immersed in IPA for 60 seconds twice. To the substrate was blown nitrogen gas for 60 seconds so that the substrate was dried.

### Experimental Examples 5-2 to 5-28

### (Preparation of Solution)

An organic matter shown in Table 5 was blended with a solvent shown in Table 5 and dissolved therein at a concentration of the organic matter as shown in Table 5.

Thus, a solution containing an organic matter and a solvent was prepared.

### (Preparation of Substrate)

In Experimental Examples 5-2 to 5-13, a substrate containing cobalt oxide (CoOx) on the surface was prepared as in Experimental Example 5-1.

In Experimental Examples 5-14 to 5-26, a silicon substrate including a 100-nm-thick cobalt film was immersed in a HF aqueous solution having a concentration of 0.5% by mass at 22° C. for one minute for removal of a natural oxide film on the surface. Thus, a substrate including a cobalt film (Co) was obtained.

In Experimental Example 5-27, the surface of a silicon substrate including a 100-nm-thick copper film was oxidized by UV/03 irradiation (lamp: EUV200WS, distance to lamp: 10 mm, ozone is generated from oxygen in the air by UV irradiation) for 30 minutes. Thus, a substrate containing copper oxide (CuOx) on the surface was obtained.

In Experimental Example 5-28, a silicon substrate including a 100-nm-thick copper film was immersed in a HF aqueous solution having a concentration of 0.5% by mass at 22° C. for one minute for removal of a natural oxide film on the surface. Thus, a substrate including a copper film (Cu) was obtained.

(Surface Treatment with Solution Containing Organic Matter)

The substrate obtained by the treatment was immersed in the solution at 22° C. for 24 hours for surface treatment of the substrate. Thus, the organic matter was deposited on the substrate surface. Then, the substrate was immersed in IPA for 60 seconds twice. To the surface of the substrate was blown nitrogen gas for 60 seconds so that the substrate was dried.

(Measurement of Contact Angle with Water)

On the substrate surface according to Experimental Examples 5-1 to 5-28 in which surface treatment with a solution containing an organic matter was carried out was put about 1 μl of pure water, and the angle (contact angle) between the water droplet and the wafer surface was measured with a contact angle meter (DM-301, available from Kyowa Interface Science Co., Ltd.) at 22° C. Table 5 shows the results.

TABLE 5

| | | | Organic matter | | Contact |
|---|---|---|---|---|---|
| | Target | | concentration | | angle |
| | surface | Organic matter | (% by mass) | Solvent | (°) |
| Experimental Example 5-1 | CoOx | n-Octadecylamine | 1 | IPA | 108 |
| Experimental Example 5-2 | CoOx | n-Hexadecylamine | 1 | IPA | 105 |
| Experimental Example 5-3 | CoOx | 4-Phenylbutylamine | 1 | IPA | 77 |
| Experimental Example 5-4 | CoOx | n-Octylamine | 1 | IPA | 95 |
| Experimental Example 5-5 | CoOx | n-Octadecylamine | 1 | EtOH | 106 |
| Experimental Example 5-6 | CoOx | n-Octadecylamine | 0.1 | PGMEA | 102 |
| Experimental Example 5-7 | CoOx | n-Hexadecylamine | 0.1 | IPA | 101 |
| Experimental Example 5-8 | CoOx | n-Hexadecylamine | 10 | IPA | 110 |
| Experimental Example 5-9 | CoOx | n-Dodecylamine | 1 | IPA | 108 |
| Experimental Example 5-10 | CoOx | n-Dodecylamine | 1 | THF | 91 |
| Experimental Example 5-11 | CoOx | n-Dodecylamine | 1 | EtOAc | 99 |
| Experimental Example 5-12 | CoOx | n-Dodecylamine | 1 | PGMEA | 98 |
| Experimental Example 5-13 | CoOx | n-Dodecylamine | 1 | anone | 65 |
| Experimental Example 5-14 | Co | n-Octadecylamine | 1 | IPA | 106 |
| Experimental Example 5-15 | Co | n-Hexadecylamine | 1 | IPA | 106 |
| Experimental Example 5-16 | Co | 4-Phenylbutylamine | 1 | IPA | 80 |
| Experimental Example 5-17 | Co | n-Octylamine | 1 | IPA | 93 |
| Experimental Example 5-18 | Co | n-Octadecylamine | 1 | EtOH | 106 |
| Experimental Example 5-19 | Co | n-Octadecylamine | 0.1 | PGMEA | 102 |
| Experimental Example 5-20 | Co | n-Hexadecylamine | 0.1 | IPA | 101 |
| Experimental Example 5-21 | Co | n-Hexadecylamine | 10 | IPA | 109 |
| Experimental Example 5-22 | Co | n-Dodecylamine | 1 | IPA | 107 |
| Experimental Example 5-23 | Co | n-Dodecylamine | 1 | THF | 90 |
| Experimental Example 5-24 | Co | n-Dodecylamine | 1 | EtOAc | 100 |
| Experimental Example 5-25 | Co | n-Dodecylamine | 1 | PGMEA | 98 |
| Experimental Example 5-26 | Co | n-Dodecylamine | 1 | anone | 67 |
| Experimental Example 5-27 | CuOx | n-Octadecylamine | 1 | IPA | 107 |
| Experimental Example 5-28 | Cu | n-Octadecylamine | 1 | IPA | 107 |

Note:
EtOH: Ethanol,
PGMEA: Propylene glycol 1-monomethyl ether 2-acetate,
THF: Tetrahydrofuran,
EtOAc: Ethyl acetate,
anone: Cyclohexanone

### Experimental Examples 6-1 to 6-15

### (Preparation of Solution)

An organic matter shown in Table 6 was blended with a solvent shown in Table 6 and dissolved therein at a concentration of the organic matter as shown in Table 6. Thus, a solution containing an organic matter and a solvent was prepared.

### (Preparation of Substrate)

In Experimental Examples 6-1 to 6-8 and 6-11 to 6-15, a silicon substrate including a 100-nm-thick silicon oxide film was immersed in a HF aqueous solution having a concentration of 0.5% by mass at 22° C. for one minute for cleaning of the surface. Thus, a substrate containing silicon oxide (SiOx) on the surface was obtained.

In Experimental Example 6-9, a silicon substrate including a 30-nm-thick silicon nitride film was immersed in a HF aqueous solution having a concentration of 0.5% by mass at 22° C. for one minute for removal of a natural oxide film on the surface. Thus, a substrate containing silicon nitride (SiN) on the surface was obtained.

In Experimental Example 6-10, a silicon substrate was immersed in a HF aqueous solution having a concentration of 0.5% by mass at 22° C. for one minute for removal of a natural oxide film on the surface. Thus, a substrate (Si substrate) containing silicon on the surface was obtained.

### (Surface Treatment)

The substrate obtained by the above treatment was immersed in the solution at 22° C. for 24 hours for surface treatment of the substrate. Thus, the organic matter was deposited on the substrate surface. Then, the substrate was immersed in IPA for 60 seconds twice. To the surface of the substrate was blown nitrogen gas for 60 seconds so that the substrate was dried.

(Measurement of Contact Angle with Water)

On the substrate surface obtained by the surface treatment according to Experimental Examples 6-1 to 6-15 was put about 1 μl of pure water, and the angle (contact angle) between the water droplet and the wafer surface was measured with a contact angle meter (DM-301, available from Kyowa Interface Science Co., Ltd.) at 22° C. Table 6 shows the results.

### Comparative Experimental Examples 1 to 6

### (Preparation of Solution)

In Comparative Experimental Examples 1, 3, and 5, an IPA solution not containing an organic matter was used as shown in Table 6.

In Comparative Experimental Examples 2, 4, and 6, trimethylsilyl dimethylamine as an organic matter was blended with PGMEA as a solvent as shown in Table 6 and dissolved therein at a concentration of the organic matter as shown in Table 6. Thus, a solution containing an organic matter and a solvent was prepared. Trimethylsilyl dimethylamine does not correspond to the organic matter of the present disclosure.

### (Preparation of Substrate)

In Comparative Experimental Examples 1 and 2, a substrate containing cobalt oxide (CoOx) on the surface was prepared as in Experimental Example 5-1. In Comparative Experimental Examples 3 and 4, a substrate including a cobalt film (Co) was prepared as in Experimental Example 5-14. In Comparative Experimental Examples 5 and 6, a substrate containing silicon oxide (SiOx) on the surface was prepared as in Experimental Example 6-1.

(Surface Treatment with Solution)

The substrate prepared in Comparative Experimental Examples 1 to 6 was immersed in the solution at 22° C. for 24 hours for surface treatment of the substrate. Then, the substrate was immersed in IPA for 60 seconds twice. To the substrate was blown nitrogen gas for 60 seconds so that the substrate was dried.

(Measurement of Contact Angle with Water)

On the substrate surface according to Comparative Experimental Examples 1 to 6 was put about 1 μl of pure water, and the angle (contact angle) between the water droplet and the wafer surface was measured with a contact angle meter (DM-301, available from Kyowa Interface Science Co., Ltd.) at 22° C. Table 6 shows the results.

TABLE 6

| | | | Organic matter | | Contact |
|---|---|---|---|---|---|
| | Target | Organic | concentration | | angle |
| | surface | matter | (% by mass) | Solvent | (°) |
| Experimental Example 6-1 | SiOx | n-Octadecylamine | 1 | IPA | 70 |
| Experimental Example 6-2 | SiOx | n-Hexadecylamine | 1 | IPA | 63 |
| Experimental Example 6-3 | SiOx | 4-Phenylbutylamine | 1 | IPA | 58 |
| Experimental Example 6-4 | SiOx | n-Octylamine | 1 | IPA | 48 |
| Experimental Example 6-5 | SiOx | n-Octadecylamine | 1 | EtOH | 68 |
| Experimental Example 6-6 | SiOx | n-Octadecylamine | 0.1 | PGMEA | 60 |
| Experimental Example 6-7 | SiOx | n-Hexadecylamine | 0.1 | IPA | 55 |
| Experimental Example 6-8 | SiOx | n-Hexadecylamine | 10 | IPA | 71 |
| Experimental Example 6-9 | SiN | n-Octadecylamine | 1 | IPA | 58 |
| Experimental Example 6-10 | Si | n-Octadecylamine | 1 | IPA | 65 |
| Experimental Example 6-11 | SiOx | n-Dodecylamine | 1 | IPA | 51 |
| Experimental Example 6-12 | SiOx | n-Dodecylamine | 1 | THF | 42 |
| Experimental Example 6-13 | SiOx | n-Dodecylamine | 1 | EtOAc | 45 |
| Experimental Example 6-14 | SiOx | n-Dodecylamine | 1 | PGMEA | 44 |
| Experimental Example 6-15 | SiOx | n-Dodecylamine | 1 | anone | 39 |
| Comparative Experimental Example 1 | CoOx | not used | 0 | IPA | 74 |
| Comparative Experimental Example 2 | CoOx | Trimethylsilyl dimethylamine | 1 | PGMEA | 75 |
| Comparative Experimental Example 3 | Co | not used | 0 | IPA | 71 |
| Comparative Experimental Example 4 | Co | Trimethylsilyl dimethylamine | 1 | PGMEA | 73 |
| Comparative Experimental Example 5 | SiOx | not used | 0 | IPA | 35 |
| Comparative Experimental Example 6 | SiOx | Trimethylsilyl dimethylamine | 1 | PGMEA | 101 |

Note:
EtOH: Ethanol,
PGMEA: Propylene glycol 1-monomethylether 2-acetate,
THF: Tetrahydrofuran,
EtOAc: Ethyl acetate,
anone: Cyclohexanone

As seen from the results in Tables 5 and 6, in the case where a solution containing an organic matter represented by the formula (1) was used and the treatment was performed using the same solution, the contact angle was larger in the case of the substrate having a surface on which a Cu oxide, a Co oxide, Cu, or Co was exposed than in the case of the substrate having a surface on which silicon, a silicon oxide, or a silicon nitride was exposed. In other words, a film of an organic matter represented by the formula (1) is formed selectively on the substrate having a surface on which a Cu oxide, a Co oxide, Cu, or Co was exposed.

In comparison of Comparative Experimental Examples 2, 4, and 6, the contact angle in Comparative Experimental Example 6 was largest, which indicates selective deposition of trimethylsilyl dimethylamine on SiOx than on a Co oxide or Co.

The organic matter represented by the formula (1) can be deposited to form a film not only on Co, Cu, an oxide of Co, and an oxide of Cu but also on a metal such as Ru, Ni, Pt, Al, Ta, Ti, and Hf or a metal oxide such as oxides of Ru, Ni, Pt, Al, Ta, Ti, and Hf which are conductive materials suitably used as wiring materials or electrode materials for semiconductor devices.

1. A selective film deposition method comprising
a step of depositing a film of an organic matter represented by the following formula (1) on a substrate having a structure where a first surface region containing at least one of a metal or a metal oxide and a second surface region containing a nonmetallic inorganic material are both exposed, selectively in the first surface region than in the second surface region, 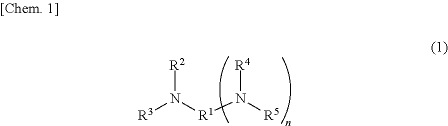
wherein N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

2. The selective film deposition method according to claim 1,
wherein a thickness t₁ of the film of an organic matter in the first surface region and a thickness t₂ of the film of an organic matter in the second surface region satisfy a ratio (t₁/t₂) of 5 or higher.

3. The selective film deposition method according to claim 1,
wherein R² and R³ in the formula (1) each represent a hydrogen atom.

4. The selective film deposition method according to claim 1,
wherein the step of depositing a film of an organic matter represented by the formula (1) selectively in the first surface region than in the second surface region is a step of exposing the substrate to an atmosphere containing the organic matter in a gaseous state.

5. The selective film deposition method according to claim 4,
wherein the organic matter is at least one selected from the group consisting of n-butylamine, n-pentylamine, n-hexylamine, n-heptylamine, n-octylamine, cyclohexylamine, aniline, ethylenediamine, and 2-aminoethanol.

6. The selective film deposition method according to claim 4,
wherein the atmosphere containing the organic matter in a gaseous state has a temperature within a range of 0° C. or higher and 200° C. or lower.

7. The selective film deposition method according to claim 4,
wherein the atmosphere containing the organic matter in a gaseous state has a pressure within a range of 13 Pa or higher and 67 kPa or lower.

8. The selective film deposition method according to claim 1,
wherein the step of depositing a film of the organic matter selectively in the first surface region than in the second surface region is a step of exposing the substrate to a solution containing the organic matter and a solvent.

9. The selective film deposition method according to claim 8,
wherein, in the formula (1), n represents 0, R² and R³ each represent a hydrogen atom, and R¹ represents a C1-C30 linear hydrocarbon group optionally containing a hetero atom or a halogen atom.

10. The selective film deposition method according to claim 9,
wherein, in the formula (1), R¹ represents a C6-C24 alkyl group.

11. The selective film deposition method according to claim 8,
wherein the organic matter is at least one selected from the group consisting of n-octylamine, n-nonylamine, n-decylamine, n-undecylamine, n-dodecylamine, n-tridecylamine, n-tetradecylamine, n-pentadecylamine, n-hexadecylamine, margarylamine, and stearylamine.

12. The selective film deposition method according to claim 8,
wherein the solution has a concentration of the organic matter represented by the formula (1) of 0.01% by mass or higher and 20% by mass or lower based on the total of the organic matter and the solvent.

13. The selective film deposition method according to claim 8,
wherein the solvent used for the solution is at least one selected from the group consisting of esters, ethers, ketones, alcoholic solvents, and polyhydric alcohol derivatives.

14. The selective film deposition method according to claim 13,
wherein the solvent used for the solution is at least one selected from the group consisting of isopropyl alcohol and ethanol.

15. The selective film deposition method according to claim 8,
wherein the substrate is washed with the solvent after selective deposition of a film of the organic matter represented by the formula (1) to the substrate.

16. The selective film deposition method according to claim 1,
wherein the metal is at least one metal selected from the group consisting of Cu, Co, Ru, Ni, Pt, Al, Ta, Ti, and Hf, and the metal oxide is an oxide of at least one metal selected from the group consisting of Cu, Co, Ru, Ni, Pt, Al, Ta, Ti, and Hf.

17. The selective film deposition method according to claim 1,
wherein the nonmetallic inorganic material is at least one selected from the group consisting of silicon, silicon oxides, silicon nitrides, and silicon oxynitrides.

18. A substrate having a structure where a first surface region containing at least one of a metal or a metal oxide and a second surface region containing a nonmetallic inorganic material are both exposed,
the substrate including a film of an organic matter represented by the following formula (1) in the first surface region,
the substrate including no film of the organic matter in the second surface region or including a film of the organic matter having a thickness t₂ smaller than a thickness t₁ of the film of the organic matter in the first surface region, 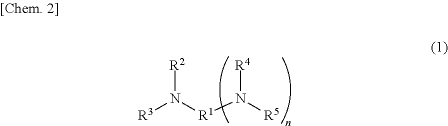
wherein N represents a nitrogen atom; R¹ represents a C1-C30 hydrocarbon group optionally containing a hetero atom or a halogen atom, R², R³, R⁴, and R⁵ each represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

19. A deposition film of an organic matter formed by the selective film deposition method according to claim 1,
the organic matter selectively deposited on a substrate being represented by the following formula (1): 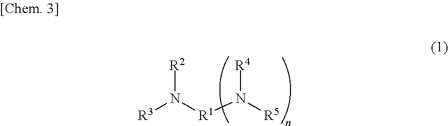
wherein N represents a nitrogen atom; R¹ represents a C1-C30 hydrocarbon group optionally containing a hetero atom or a halogen atom, R², R³, R⁴, and R⁵ each represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

20. An organic matter to be used in the selective film deposition method according to claim 1,
the organic matter being represented by the following formula (1): 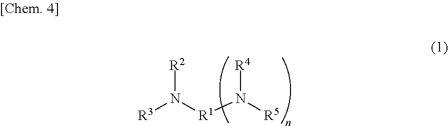
wherein N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

21. A solution comprising:
an organic matter represented by the following formula (1); and
a solvent, 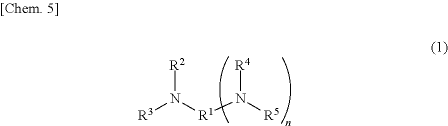
wherein N represents a nitrogen atom; R¹ represents a C1-C30 hydrocarbon group optionally containing a hetero atom or a halogen atom, R², R³, R⁴, and R⁵ each independently represent a hydrogen atom or a C1-C10 hydrocarbon group optionally containing a hetero atom or a halogen atom, where the hydrocarbon group covers a branched or cyclic hydrocarbon group when containing 3 or more carbon atoms; and n represents an integer of 0 or larger and 5 or smaller, where n representing 0 gives a case where R⁴ and R⁵ are not present.

22. The solution according to claim 21,
wherein the organic matter is at least one selected from the group consisting of n-octylamine, n-nonylamine, n-decylamine, n-undecylamine, n-dodecylamine, n-tridecylamine, n-tetradecylamine, n-pentadecylamine, n-hexadecylamine, margarylamine, and stearylamine,
the solvent is at least one selected from the group consisting of ethanol and isopropyl alcohol, and
the solution has a concentration of the organic matter represented by the formula (1) of 0.01% by mass or higher and 20% by mass or lower based on the total of the organic matter and the solvent.
